(19) United States
(12) Patent Application Publication
(10) Pub. No.: US 2022/0101959 A1
(43) Pub. Date: Mar. 31, 2022

(54) CLINICAL TRIALS MATCHING AND VISUALIZATION IN A CLINICAL GUIDELINE VIEW

(71) Applicant: Siemens Healthcare GmbH, Erlangen (DE)

(72) Inventors: Lisa VALLINES, Weisendorf (DE); Benedikt KRUEGER, Ebensfeld (DE); Aditya MAHARA, Astoria, NY (US)

(73) Assignee: Siemens Healthcare GmbH, Erlangen (DE)

(21) Appl. No.: 17/463,707

(22) Filed: Sep. 1, 2021

(30) Foreign Application Priority Data
Sep. 29, 2020 (DE) 10 2020 212 237.7

(51) Int. Cl. G16H 10/20; G16H 10/60; G16H 40/20

(52) U.S. Cl. CPC G16H 10/20; G16H 40/20; G16H 10/60

(57) ABSTRACT

A medical recommendation system includes a computing device. The device is configured to implement a patient data receiving module configured to receive medical patient data pertaining to a patient; to implement a clinical trial data receiving module configured to receive clinical trial data; and to implement a medical guideline receiving module configured to receive a medical guideline in a basic version, represented by a basic connected directed graph (BCDG) including a plurality of nodes representing medical actions; to implement a guideline adapting module configured to determine whether the patient fulfils criteria of at least one clinical trial based on the received medical patient data and the received clinical trial data and to adapt, upon the patient fulfilling the criteria, the BCDG to provide an adapted connected directed (ACDG) graph by integrating at least one additional node into the BCDG; and to implement an output module configured to output the ACDG.

### PRIORITY STATEMENT

The present application hereby claims priority under 35 U.S.C. § 119 to German patent application number DE 102020212237.7 filed Sep. 29, 2020, the entire contents of which are hereby incorporated herein by reference.

### FIELD

Example embodiments of the invention generally relate to a medical recommendation system and a method for providing a medical recommendation.

### BACKGROUND

In the technical field of healthcare, clinical trials are experiments or observations done in clinical research. Prospective biomedical or behavioural research studies on patients are designed to answer specific questions on biomedical or behavioural treatments, including new treatments such as novel vaccines, drugs, dietary choices, dietary supplements or medical devices and known treatments that require further study and comparison. Clinical trials generate data on safety and efficacy. Typically, they are only conducted after they have received health authority approval in the country where the health authority approval is sought. Health authorities are responsible for vetting a risk-benefit ratio of the clinical trials. A health authority approval for a biomedical or behavioural treatment does not automatically mean that the biomedical or behavioural treatment is effective.

Depending on product type and development stage of the biomedical or behavioural treatment, researchers initially enrol volunteers or patients into small pilot clinical trials, and subsequently conduct progressively larger scale comparative clinical trials. The clinical trials can vary in size and cost, and they can involve a single research centre or multiple research centres. The clinical trials can be conducted in one country or in multiple countries. Clinical trial study design aims to ensure scientific validity and reproducibility of results of the clinical trials.

Costs for the clinical trials can range into billions of dollars per approved biomedical or behavioural treatment. Sponsors for the clinical trials may be for example governmental organizations, pharmaceutical companies, biotechnology device companies or medical device companies. Certain activities necessary to the clinical trials, such as monitoring and lab work, may be managed by an outsourced partner, such as a contract research organization or a central laboratory.

Enrolling a patient into a clinical trial conducted by a third party can have financial benefits for hospitals and additionally or alternatively medical professionals. Further, if the patient has limited or no options in standard clinical treatments, enrolling the patient in a suitable clinical trial may increase a survival probability of the patient or improve the course of a disease.

However, for the medical professionals as well as for the patient it is difficult to determine whether the patient is eligible for the clinical trial.

For medical professionals it is very time consuming to determine whether and under which conditions each patient is eligible for the clinical trial. In particular, it is difficult for the medical professional to determine whether

the patient is eligible for the clinical trial,

the patient is not eligible for the clinical trial, or

it cannot be determined whether the patient is eligible or not eligible for the clinical trial based on the current patient data.

The effort for determining an eligibility for the clinical trial increases accordingly for determining an eligibility for a plurality of clinical trials. Moreover, the eligibility of a clinical trial may itself be subject to preconditions which themselves again have various eligibility criteria, and so on.

Further, the patient is interested in receiving information related to suitable clinical trials, corresponding consequences of enrolling into the clinical trial, and possible consequences for the course of the disease.

Thus, for the medical professional the task of determining eligibility of a patient with regard to one or more available trials may be overwhelming, wherein the complex interactions between conditions, conditioned options and so on following different choices, outcomes of tests (which may themselves be conditioned) and so on may be simply unresolvable for the professional.

### SUMMARY

At least one embodiment of the present invention improves the human-machine interaction between users and a medical recommendation system, in particular regarding easy and reliable enrolment of patients in clinical trials. Thereto, embodiments of the present invention provide a medical recommendation system and a method for providing a medical recommendation. Additional advantageous features, embodiments and/or variants of embodiments are described in the claims and the specification.

According to a first embodiment of the present invention a medical recommendation system for a user comprises a computing device. The computing device is configured to implement a patient data receiving module configured to receive medical patient data pertaining to a patient. Further, the computing device is configured to implement a clinical trial data receiving module configured to receive clinical trial data. The computing device is also configured to implement a medical guideline receiving module configured to receive a medical guideline in a basic version. The medical guideline is represented by a basic connected directed graph (BCDG) comprising a plurality of nodes representing medical actions and a plurality of directed edges representing options and additionally or alternatively possibilities. Furthermore, the computing device is configured to implement a guideline adapting module configured to determine whether the patient fulfils predetermined criteria of at least one clinical trial based on the received medical patient data and the received clinical trial data. The guideline adapting module is also configured to adapt, if the patient fulfils the predetermined criteria of the at least one clinical trial, the BCDG to provide an adapted connected directed (ACDG) graph by integrating at least one additional node into the BCDG. The computing device is moreover configured to implement an output module configured to output the ACDG.

According to a second embodiment of the present invention a method for providing a medical recommendation to a user, comprises:

Receiving medical patient data pertaining to a patient;

Receiving clinical trial data;

Receiving a medical guideline in a basic version. The medical guideline is represented by a basic connected directed graph comprising a plurality of nodes representing medical actions and a plurality of directed edges representing options and additionally or alternatively possibilities;

Determining whether the patient fulfils predetermined criteria of at least one clinical trial based on the received medical patient data and the received clinical trial data and adapting, if the patient fulfils the predetermined criteria of the at least one clinical trial, the basic connected directed graph to provide an adapted connected directed graph by integrating at least one additional node into the basic connected directed graph; and

Outputting the adapted connected directed graph.

According to a third embodiment of the present invention, a computer program product comprises instructions (i.e. executable program code) which, when the program is executed by a computer, cause the computer to carry out the steps of the method according to the second embodiment of the present invention.

According to a fourth embodiment of the present invention, a computer-readable data carrier has stored thereon the computer program product according to the third embodiment of the present invention.

According to a fifth embodiment of the present invention, a data carrier signal carries the computer program product according to the third embodiment of the present invention. In other words, a data stream is provided which comprises, or is configured to generate, executable program code configured to perform, when executed, the steps according to any embodiment of the method according to the first embodiment of the invention.

According to an embodiment of the present invention, a medical recommendation system for a user, comprises:

a computing device, configured to implement at least:
a patient data receiving module configured to receive medical patient data pertaining to a patient;
a clinical trial data receiving module configured to receive clinical trial data;
a medical guideline receiving module, configured to receive a medical guideline in a basic version, the medical guideline being represented by a basic connected directed graph (BCDG) including a plurality of nodes representing medical actions and a plurality of directed edges representing at least one of options and possibilities;
a guideline adapting module configured to determine whether the patient fulfils criteria of at least one clinical trial based on the medical patient data received and the clinical trial data received, and configured to adapt the BCDG, upon the patient being determined to have fulfilled the criteria of the at least one clinical trial, to provide an adapted connected directed graph (ACDG) by integrating at least one additional node into the BCDG; and
an output module configured to output the ACDG.

According to an embodiment of the present invention, a method for providing a medical recommendation to a user, comprises:

receiving medical patient data pertaining to a patient;

receiving clinical trial data;

receiving a medical guideline in a basic version, the medical guideline being represented by a basic connected directed graph (BCDG) including a plurality of nodes representing medical actions and a plurality of directed edges representing at least one of options and possibilities;

determining whether the patient fulfils criteria of at least one clinical trial based on the medical patient data received and the clinical trial data received and adapting the BCDG, upon the determining indicating that the patient fulfils the criteria of the at least one clinical trial, to provide an adapted connected directed graph (ACDG) by integrating at least one additional node into the BCDG; and

outputting the ACDG.

According to an embodiment of the present invention, a non-transitory computer program product stores executable program code which, when executed by a computer, cause the computer to carry out the method of an embodiment.

According to an embodiment of the present invention, a non-transitory computer-readable data carrier stores executable program code which, when executed by a computer, cause the computer to carry out the method of an embodiment.

### BRIEF DESCRIPTION OF THE DRAWINGS

The invention will be explained in greater detail with reference to example embodiments depicted in the drawings as appended.

The accompanying drawings are included to provide a further understanding of the present invention and are incorporated in and constitute a part of this specification. The drawings illustrate the embodiments of the present invention and together with the description serve to explain the principles of the invention. Other embodiments of the present invention and many of the intended advantages of the present invention will be readily appreciated as they become better understood by reference to the following detailed description. Like reference numerals designate corresponding similar parts.

FIG. 1 shows a schematic diagram illustrating a medical recommendation system according to an embodiment of the present invention;

FIG. 2 shows a schematic diagram illustrating a medical recommendation system according to another embodiment of the present invention;

FIG. 3 shows an adapted connected directed graph according to an embodiment of the present invention;

FIG. 4 shows an adapted connected directed graph according to another embodiment of the present invention;

FIG. 5 shows an adapted connected directed graph according to another embodiment of the present invention;

FIG. 6 shows an adapted connected directed graph according to another embodiment of the present invention;

FIG. 7 shows an embodiment of a medical professional or patient computing device;

FIG. 8 shows a schematic flow diagram illustrating a method according to an embodiment of the second embodiment of the present invention;

FIG. 9 schematically illustrates a computer program product according to an embodiment of the third embodiment of the invention; and

FIG. 10 schematically illustrates a non-transient computer-readable data storage medium according to an embodiment of the fourth embodiment of the invention.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. At least one embodiment of the present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

According to a first embodiment of the present invention a medical recommendation system for a user comprises a computing device. The computing device is configured to implement a patient data receiving module configured to receive medical patient data pertaining to a patient. Further, the computing device is configured to implement a clinical trial data receiving module configured to receive clinical trial data. The computing device is also configured to implement a medical guideline receiving module configured to receive a medical guideline in a basic version. The medical guideline is represented by a basic connected directed graph (BCDG) comprising a plurality of nodes representing medical actions and a plurality of directed edges representing options and additionally or alternatively possibilities. Furthermore, the computing device is configured to implement a guideline adapting module configured to determine whether the patient fulfils predetermined criteria of at least one clinical trial based on the received medical patient data and the received clinical trial data. The guideline adapting module is also configured to adapt, if the patient fulfils the predetermined criteria of the at least one clinical trial, the BCDG to provide an adapted connected directed (ACDG) graph by integrating at least one additional node into the BCDG. The computing device is moreover configured to implement an output module configured to output the ACDG.

According to a second embodiment of the present invention a method for providing a medical recommendation to a user, comprises:

Receiving medical patient data pertaining to a patient;

Receiving clinical trial data;

Receiving a medical guideline in a basic version. The medical guideline is represented by a basic connected directed graph comprising a plurality of nodes representing medical actions and a plurality of directed edges representing options and additionally or alternatively possibilities;

Determining whether the patient fulfils predetermined criteria of at least one clinical trial based on the received medical patient data and the received clinical trial data and adapting, if the patient fulfils the predetermined criteria of the at least one clinical trial, the basic connected directed graph to provide an adapted connected directed graph by integrating at least one additional node into the basic connected directed graph; and

Outputting the adapted connected directed graph.

According to a third embodiment of the present invention, a computer program product comprises instructions (i.e. executable program code) which, when the program is executed by a computer, cause the computer to carry out the steps of the method according to the second embodiment of the present invention.

According to a fourth embodiment of the present invention, a computer-readable data carrier has stored thereon the computer program product according to the third embodiment of the present invention.

According to a fifth embodiment of the present invention, a data carrier signal carries the computer program product according to the third embodiment of the present invention. In other words, a data stream is provided which comprises, or is configured to generate, executable program code configured to perform, when executed, the steps according to any embodiment of the method according to the first embodiment of the invention.

The user may be, for example, a medical professional and/or the patient. Other users, for example an administrative employee or a technical assistant tasked with operating a medical device may also be possible.

The computing device, as the term is used herein, may be understood to be any means for computing, i.e. for running a software or an algorithm. For example, the computing device may comprise a single processor core (e.g. central processing unit CPU, graphical processing unit GPU, etc.) or a microprocessor and working memory (e.g. random access memory RAM). The computing device may comprise an array of processor cores or central processing units (CPUs). The computing device may also comprise an array of graphical processing units (GPUs). In particular, the computing device may be a stationary computer (e.g. PC, terminal, etc.) or a mobile device (laptop, tablet, smartphone, etc.). The computing device may be realized partially or completely by interconnected remote devices such as cloud computing servers and additionally or alternatively as a virtual machine.

The computer-readable data carrier may comprise, or consist of, any type of computer memory, in particular semiconductor memory such as a solid-state memory. The data storage medium may also comprise, or consist of, a CD, a DVD, a Blu-Ray-Disc, a USB memory stick or the like.

The medical patient data contains medical information about a patient. The medical information provides information about the medical state and/or medical history of the respective patient and can be the basis for a diagnosis. For example, the medical patient data may comprise personal data (e.g. gender, age, ethnicity, height, weight, body mass index, etc.), medical history data (e.g. current diagnoses, earlier diagnoses, heredopathia, interventions, treatments, medication, etc.), medical image data, lab test results, pathology data, genetic data, and the like.

The medical patient data may be received from or provided by any medical patient data source that is capable of providing medical patient data. In particular, each medical patient data source may represent, or be connected to, a different source of medical patient data. For example, a medical patient data source may be a medical imaging device (MRI scanner, CT scanner, ultrasound device, PET scanner, etc.), a laboratory apparatus, DNA sequencer, hospital management system, patient files (paper files or digital files), and the like.

Some or all of the medical patient data sources may be data interfaces that connect the medical recommendation system to external or internal data storage sections holding the medical patient data, wherein the data interfaces may be realized in hardware and additionally or alternatively software. For example, a first data interface may be a software interface adapted to access a database with (some or all of) the medical patient data, a second data interface may comprise a data cable linking the computing device to an imaging device (such as an MRI device), a third data interface may be a software agent adapted to retrieve medical patient data from the internet and additionally or alternatively a remote data depository or a distributed data depository, and so on. The patient, or a device operated or owned by the patient, may itself be a medical patient data source.

The clinical trial data comprises information on predetermined criteria that the patient has to fulfil in order to be eligible for the at least one clinical trial. The predetermined criteria may be specific medical parameters that have to be met. For example, the predetermined criteria may include a maximum age of the patient, a specific blood type, a specific course of disease, and the like. Furthermore, the clinical trial data may comprise, for example, information regarding the course of the at least one clinical trial (in particular, medical actions that has to be performed by the patient and/or the physician due to the participation at the clinical trials, e.g. diagnostic or therapeutic actions, in particular, using a certain drug with the patient or using a certain drug with the patient in a certain way), the duration of the at least one clinical trial and additionally or alternatively the geographic location of sites where the clinical trial is conducted.

The predetermined criteria may be sufficient and/or necessary criteria/conditions or any combination thereof. Thus, meeting all of the predetermined criteria may not be equivalent to being eligible for a trial, because, for example, all of the predetermined criteria that are evaluated by the guideline adapting module may be necessary (but not sufficient) conditions. Checking for eligibility of a patient may then comprise evaluation additional, sufficient conditions/criteria which may also be comprised within the clinical trial data. If in this way multiple sets of criteria are present (e.g. a first set of necessary conditions and a second set of sufficient conditions or the like), then the guideline adapting module may be configured to first evaluate only a first set of multiple sets of predetermined criteria.

The clinical trial data may be received from any clinical trial data source that is capable of providing clinical trial data. In particular, the clinical trial data may be received from a database owned by a research centre or may be received from a database owned by a plurality of research centres. The plurality of research centres may be located in one country or may be located in a plurality of countries. A research centre may for example be or be owned by a governmental organization, a pharmaceutical company or a biotechnology company. Optionally, the clinical trial data may also be received from an outsourced partner, such as a contract research organization or a central laboratory.

The medical guideline provides a course of action for providing medical care to patients having a specific condition, specific symptoms, or a specific illness. Thereto, the medical guideline prescribes a sequence of medical actions. A medical action may be a diagnosis step (e.g. anamneses, medical tests, medical imaging, etc.), a therapy step (e.g. medication, intervention, etc.), or a decision-making process. A possible medical action can be, for example, an MRI examination, a PET scan, an ultrasound examination, a physical therapy, a deduction diagnosis, a coronary intervention, a diabetes medication, or the like. A medical action may comprise a sequence of medical steps.

Each medical step of the sequence of medical steps may be an interaction of a medical professional with a medical device or fully automated medical steps performed by the medical device. For example, in the step MRI examination, the medical steps of aligning the patient in the MRI scanner, selecting a pulse sequence based on the medical patient information, executing the pulse sequence with the MRI scanner, injecting contrast agent into the patient, selecting a pulse sequence based on the medical patient information and injected contrast agent, executing the pulse sequence with the MRI scanner, and the like are executed by medical staff operating, i.e. interacting with, the MRI scanner.

The medical guideline in the basic version may be a predefined, standardized course of action. The medical guideline in the basic version may be a medical guideline provided by a governmental body, a medical organisation or a hospital. The medical guideline in the basic version may be chosen as prescribed course of action in the hospital in which the medical recommendation system is operated.

A medical guideline is represented by a connected directed graph comprising a plurality of at least two nodes and a plurality of at least one directed edge, where medical actions are represented by nodes and options and additionally or alternatively possibilities are represented by directed edges. Each node can be connected to a successive node via a directed edge. Each directed edge connects two nodes in one direction. For example, from a node representing tumor staging via PET imaging successive nodes representing radio therapy, resection, chemotherapy, and combined therapies can be reached via respective edges depending on the outcome of the tumor staging. Consequently, a patient (or the treatment status of the patient) can proceed from node to node along the directed edges through the connected directed graph according to the respective medical guideline during treatment of a specific condition/symptoms/illness.

Each node of the plurality of nodes is connected to (at least) another node of the plurality of nodes via a directed edge of the plurality of edges. The options that are represented by the plurality of edges may be decision options. For example, a current position of the patient in the medical guideline may be indicated by an icon showing a human figure. Starting from the current position of the patient the medical professional or the patient may be able to choose between at least two medical actions, therefore having a plurality of options. The possibilities represented by the directed edges may also be referred to as weighted options. The possibilities may comprise a factor indicating a choice recommendation. The choice recommendation may for example indicate a disease recovery probability indicating the probability with which a recovery is to be expected, when choosing the respective option, e.g. treatment, intervention, clinical trial, etc. The weighting of several options may be graphically represented for example by drawing an edge with a comparatively thick line when it is recommended to a higher degree, and with a comparatively thin line when it is recommended to a lower degree.

The medical guideline in the basic version is represented by the basic connected directed graph (BCDG). Each medical action of the medical guideline in the basic version is represented by a node of the BCDG and each option and possibility of the medical guideline in the basic version is represented by a directed edge of the BCDG.

The guideline adapting module is configured to determine whether a patient fulfils the predetermined criteria by comparing (or: evaluating) the received medical patient data and the received clinical trial data. The received clinical trial data may comprise the predetermined criteria of the at least one clinical trial. The predetermined criteria may for example be a maximum age of the patient or a specific clinical pattern of the patient.

The basic connected directed graph (BCDG) represents the medical guideline in the basic version, for example, as received from the hospital in which the medical guideline in the basic version is practised and in which the medical recommendation system is operated. In case, the patient fulfils the predetermined criteria i.e. is eligible for entering the at least one clinical trial the guideline adapting module adapts the BCDG such that the at least one additional node, i.e. medical action, becomes available. The thus available additional medical action may be entering the at least one clinical trial or conducting a further medical examination/diagnosis step. The adapted BCDG including the option or possibility to choose the at least one additional node (medical action such as entering the at least one clinical trial) is the adapted connected directed graph (ACDG). The ACDG, thus, represents the medical guideline after integrating the at least one additional medical action into the medical guideline in the basic version.

The ACDG may further comprise at least one additional edge for connecting the at least one additional node to its respective preceding node. An additional edge of the at least one additional edge may for example connect a node of the plurality of nodes (of the BCDG) with an additional node of the at least one additional node (new in the ACDG). At least one node of the at least one node may represent the at least one clinical trial. Alternatively or additionally, at least one node of the at least one node may represent an additional medical action to test an eligibility of the patient for the at least one clinical trial. In some cases, the at least one additional node may also be inserted within an already existing edge, thereby splitting the edge into two new edges.

In particular, the guideline adapting module can utilize the received clinical trial data in order to determine the at least one additional node to include into the BCDG, and the position where to enter the at least one additional node into the BCDG (i.e., the existing nodes within the BCDG the additional node needs to be connected with). In particular, the received clinical trial data can comprise structured information about actions to be executed for a patient within the clinical trial, e.g. in the form of a directed graph and/or XML data (acronym for “Extensible Markup Language”). In particular, the structured information can additionally specify how the actions to be performed relate to an existing medical guideline. It is also possible that the received clinical trial data comprises unstructured data (e.g., narrative text that describes the actions to be taken for a patient within the clinical study). In particular, this unstructured data can be analysed by the guideline adapting module using natural language processing or other known methods for analysing unstructured information.

With at least one embodiment of the present invention, medical practitioners and patients are guided in an objectively better manner such as to easily and reliably chose the further course of action in treatment or in providing medical care from all possible further medical actions including medical trials without having to switch between different devices or platforms.

In some advantageous embodiments, the medical recommendation system comprises at least one medical professional graphical user interface configured to display at least a part of the ACDG (or, preferably, the entire ACDG) to a medical professional. Different parts of the ACDG may be display depending on, for example, a qualification of the medical professional. For example, nodes representing decisions to be taken only by a very experience specialist may only be displayed, together with the edges leading to/from that node, to a medical professional with the necessary qualification. The qualifications may be entered in a certified manner in a user account of each medical professional.

The ACDG may also be displayed partially and/or completely based on settings of a user, e.g. of a medical professional. For example, all edges/nodes relating to procedures of a certain type may be selected not to be displayed (for example, all edges/nodes representing procedures that cannot be performed in a hospital in which the patient is currently registered, or the like).

The at least one medical professional graphical user interface may be displayed on a monitor of, or operatively coupled to, the computing device of the medical recommendation system. Alternatively, the medical professional graphical user interface may be displayed on a monitor of another computing device that is communicatively connected with the computing device of the medical recommendation system, in particular with the output module.

In this way the medical professional can view the ACDG and therefrom immediately grasp the current medical action/node the patient is in and the possible further medical actions/nodes the patient can be subjected to. In particular, the medical professional can easily and reliably decide how to proceed in providing medical care to the patient. The system according to at least one embodiment of the invention thus provides the user with insight into the complex internal medical state of the patient, as well as into the status of the patient within the vast and complex network of possible treatments, medical devices, and clinical trials.

In some advantageous embodiments, the medical recommendation system comprises at least one patient graphical user interface configured to display the ACDG, or at least a part of the ACDG, to the patient. Nodes and/or edges of the BCDG or the ACDG may be provided with metadata indicating whether or not the respective node and/or edge is/are to be displayed to the patient. The ACDG may also be provided with specific nodes and/or edges generated specifically for the patient, for example nodes indicating choices or decisions to be made by the patient and/or the like.

The at least one patient graphical user interface may be displayed on a monitor of the computing device of, or operatively coupled to, the medical recommendation system. Alternatively, the patient graphical user interface may be displayed on a monitor of another computing device that is communicatively connected with the computing device of the medical recommendation system, in particular with the output module.

In this way the patient can view the ACDG and therefrom understand the current medical action/node they are in and the possible further medical actions/nodes they can be subjected to. In particular, the patient can easily and reliably decide how to proceed in receiving medical care.

In some advantageous embodiments, at least one node of the at least one additional node represents an additional medical action to test an eligibility of the patient for the at least one clinical trial.

In case the patient could fulfil the predetermined criteria of the at least one clinical trial depending on the outcome or result of a further medical test, the at least one additional node of the ACDG, which additional node is integrated into the BCDG, may represent the additional medical action to test the eligibility of the patient for the at least one clinical trial. For example, in case a certain tumor staging or pathological examination of the tumor is needed to determine whether the patient is eligible for the at least one clinical trial, a respective node of conducting the necessary medical test is introduced to the BCDG.

This is particularly useful when specific medical patient data that has not been collected and that requires a medical examination is required to evaluate the eligibility of the patient for the at least one clinical trial. Thus, the patient and the medical professional are reliably informed about possible clinical trials and the further medical examination(s) needed for determining eligibility of the patient for the clinical trial.

In some advantageous embodiments, at least one node of the at least one additional node represents the at least one clinical trial.

The node representing the at least one clinical trial may in particular represent an entry into the respective clinical trial. The clinical trial as such may be conducted at a different site. Additionally, the medical steps of the clinical trial may also be included in the node representing the at least one clinical trial.

In case all medical patient data needed for determining eligibility of the patient for the study are present, the at least one additional node can be the clinical trial without any nodes of clinical examinations arranged before the node of the clinical trial. In case some medical patient data needed for determining eligibility of the patient for the study is missing, there is at least one node representing the respective medical examination present before the at least one node of clinical examination.

This is particularly advantageous because the basic clinical guideline, eligible clinical trials, and potentially eligible clinical trials are simultaneously displayed to the medical professional/patient. Thus, all available information about optional/possible further medical actions is easily and reliably available to the medical professional/patient. The described manner of presenting this information is objectively better than previous attempts as it can capture the full logical/causal dependency structure of interrelated medical actions, treatments, states, conditions, choices, and so on.

In some advantageous embodiments, at least one node of the plurality of nodes representing the medical actions comprises a sequence of medical steps. Each of the medical steps may, for example, represent an interaction of the medical professional with a medical device. The guideline adapting module may be configured to modify the at least one node of the plurality of nodes by adding at least one further medical step to and additionally or alternatively or replacing at least one medical step in the sequence of medical steps.

As described above, a medical action represented by a node in the BCDG and ACDG may comprise several medical steps. For example, in the medical action “haemogram” represented by a respective node in the BCDG a complete blood count (CBC) without differential is determined.

The guideline adapting module is configured to adapt the BCDG into the ACDG by adding at least one further medical step to the sequence of medical steps (modified node). Further, the guideline adapting module is configured to replace at least one medical step in the sequence of medical steps (modified node) in order to adapt the BCDG into the ACDG. For example, in the medical action “haemogram” represented by the respective modified node in the ACDG the additional medical step “differential” is added such that in the corresponding adapted guideline a CBC with differential is determined.

This is particularly advantageous because this results in a leaner graphical visualization of the adapted connected directed graph. Leaner in this context means that less additional nodes are necessary to adapt the BCDG into the ACDG.

In some advantageous embodiments, the guideline adapting module is configured to generate an examination request signal. The examination request signal indicates that at least one additional medical examination of the patient is required to identify an eligible clinical trial. The output module is configured to output the examination request signal to the at least one medical professional graphical user interface and additionally or alternatively to the at least one patient graphical user interface.

The examination request signal is a signal that includes information about which additional medical examination, i.e. which medical action including which medical steps, is required to determine eligibility of the patient for the possibly eligible clinical trial.

In this way the medical professional and additionally or alternatively the patient can be directly and reliably notified that at least one additional medical examination of the patient is required to identify an eligible clinical trial.

In some advantageous embodiments, the computing device further implements an information request module that is configured to generate an information request signal. The information request signal indicates that additional medical patient data is required. The output module is configured to output the information request signal to the at least one medical professional graphical user interface and additionally or alternatively to the at least one patient graphical user interface.

For example, the medical patient data of the patient already provided may be a set of information comprising the age of the patient, the sex of the patient and the previous diagnoses of the patient. At least one clinical trial of the at least one clinical trial may for example only be open to patients with a specific blood type. Therefore, the specific blood type is one of the predetermined criteria the patient has to fulfil in order to enter the clinical trial (i.e. a necessary condition). The information request module thus generates the information request signal indicating that additional information on the blood type of the patient, i.e. (at least one type of) additional medical patient data, is required. The at least one medical professional graphical user interface and additionally or alternatively or the at least one patient graphical user interface may therefore output a corresponding notice to at least one medical professional or to the patient.

This is particularly useful when specific medical patient data that has not been collected is required to evaluate the eligibility of the patient for the at least one clinical trial.

In some advantageous embodiments, the additional medical patient data comprises at least one of the following:

information about a medical history of the patient;

information about pre-existing medical conditions of the patient,

information indicating a distance the patient is willing to travel to participate in a clinical trial and

information indicating a preferred type of therapy.

The additional medical patient data is medical patient data as described above that is added after the user (medical professional/patient) has been prompted to provide it. Thereby, the medical history may for example comprise, or consists of, previous diagnoses, prior lines of therapy, and/or the like. The pre-existing medical conditions may for example be a stage of cancer, common somatic mutations, comorbidities, and the like. The information indicating a distance may also be a time of travel or expenses for the travel the patient is willing or able to spend. The information indicating a preferred type of therapy may be used to automatically emphasize eligible clinical trials for therapies that correspond to the preferred type.

With the additional medical patient data determination of an eligible clinical trial can be particularly fast and reliably effected.

In some advantageous embodiments, the at least one patient graphical user interface is configured to enable the patient to enter the additional medical patient data. The patient data receiving module is configured to receive the additional medical patient data. The patient data receiving module is configured to add the entered additional medical patient data to the medical patient data.

The patient graphical user interface, as described above, may be displayed on a monitor of the computing device of the medical recommendation system or on a monitor of another computing device that is communicatively connected with the computing device of the medical recommendation system. Based on the information displayed to the patient via the patient graphical user interface the patient may enter the needed additional medical patient data via an input device (e.g. keyboard, mouse, touchscreen, interface etc.) connected with the computing device of the medical recommendation system or with the other computing device that is communicatively connected with the computing device of the medical recommendation system.

If, for example, the information request signal is outputted to the patient via the at least one patient graphical user interface, the patient may be able to enter the additional medical patient data by typing the respective missing information on a keyboard or providing corresponding files including the additional medical patient data from a USB stick or the like. The patient data receiving module may receive the entered additional medical patient data, e.g. the blood type of the patient, and add the entered additional medical patient data to the medical patient data of the patient. An example set of already provided medical patient data comprising the age of the patient, the sex of the patient and the previous diagnoses of the patient can for example be extended by the additional medical patient data of the blood type of the patient.

In this way the further medical information about the patient needed to decide whether he is eligible for a certain clinical trial can be provided in a particularly easy and fast manner.

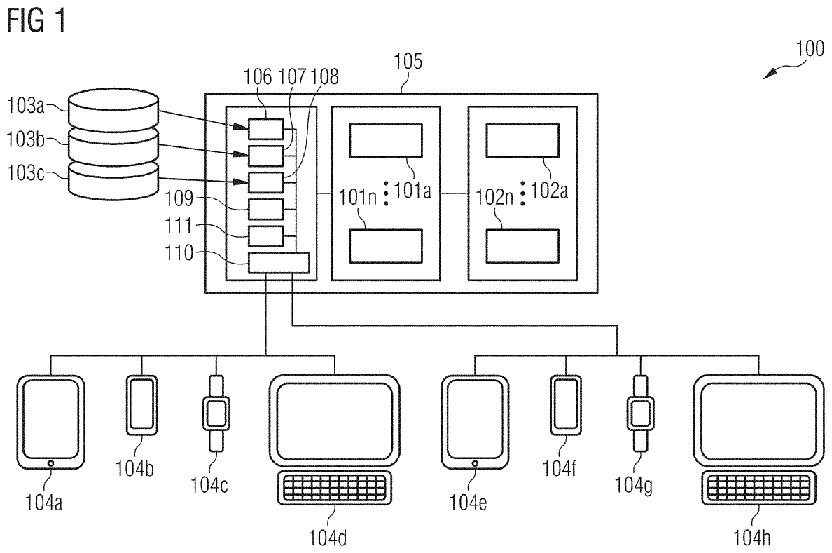

FIG. 1 shows a schematic diagram illustrating a medical recommendation system 100 for a user.

The medical recommendation system 100 comprises a computing device 105. The computing device 105 of the example shown in FIG. 1 implements a patient data receiving module 106, a clinical trial data receiving module 107, a medical guideline receiving module 108, a guideline adapting module 109, an output module 110 and an information request module 111. Further, the computing device 105 comprises at least one processor 101a-n and at least one memory 102a-n.

As shown in FIG. 1 the patient data receiving module 106 is connected to a medical patient data source 103a, the clinical trial data receiving module 107 is connected to a clinical trial data source 103b and the medical guideline receiving module 108 is connected to a medical guideline data source 103c. The output module 110 is connected to at least one medical professional computing device 104a-d configured to display a medical professional graphical user interface and to at least one patient computing device 104e-h configured to display a patient graphical user interface. The at least one medical professional computing device 104a-d and the at least one patient computing device 104e-h may also be connected to the data receiving module 106.

In FIG. 1, in order to provide some examples, the medical professional computing device 104a is a tablet computer, the medical professional computing device 104b is a smartphone, the medical professional computing device 104c is a smart watch and the medical professional computing device 104d is a personal computer, also referred to as PC. Furthermore, the patient computing device 104e is a tablet computer, the patient computing device 104f is a smartphone, the patient computing device 104g is a smart watch and the patient computing device 104h is a personal computer, also referred to as PC. The at least one medical professional computing device 104a-d and additionally or alternatively or the at least one patient computing device 104e-h may be considered part of the medical recommendation system 100 or may not be considered part of the medical recommendation system 100.

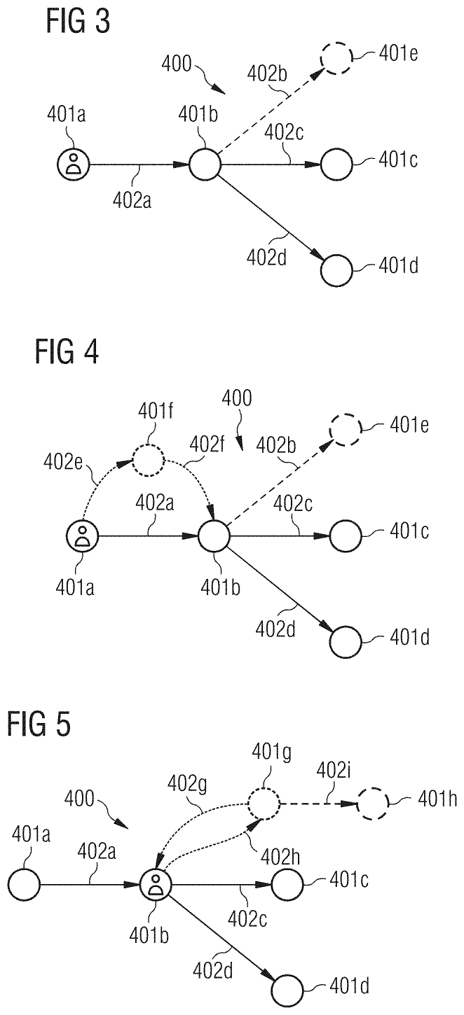

The patient data receiving module 106 in FIG. 1 is configured to receive medical patient data pertaining to a patient from the medical patient data source 103a. The clinical trial data receiving module 107 is configured to receive clinical trial data from the clinical trial data source 103b. The medical guideline receiving module 108 is configured to receive a medical guideline in a basic version from the medical guideline data source 103c. The medical guideline is represented by a basic connected directed graph (BCDG) comprising a plurality of nodes 401a-q representing medical actions and a plurality of directed edges representing options and additionally or alternatively possibilities (see also FIGS. 3-6).

After receiving the medical patient data, the clinical trial data and the medical guideline in the basic version, the guideline adapting module 109 determines whether the patient fulfils predetermined criteria of at least one clinical trial based on the received medical patient data from the medical patient data source 103a and the received clinical trial data from the clinical trial data source 103b. If, for example, the medical recommendation system 100, in particular the guideline adapting module 109, determines that at least one additional medical examination of the patient is required to identify an eligible clinical trial, the guideline adapting module 109 may generate an examination request signal indicating that the at least one additional medical examination of the patient is required. The output module 110 then outputs the examination request signal to the at least one medical professional computing device 104a-d for displaying via the medical professional graphical user interface and additionally or alternatively to the at least one patient computing device 104e-h via the patient graphical user interface.

Additionally or alternatively, if the medical recommendation system 100, in particular the guideline adapting module 109, determines that additional medical patient data is required to identify an eligible clinical trial, the information request module 111 generates an information request signal indicating that the additional medical patient data is required. The output module 110 then outputs the information request signal to the at least one medical professional computing device 104a-d for displaying via the medical professional graphical user interface and additionally or alternatively to the at least one patient computing device 104e-h via the patient graphical user interface.

After receiving the information request signal from the information request module 111 the at least one patient computing device 104e-h enables the patient via the patient graphical user interface to enter the additional medical patient data, for example, via a keyboard or a USB-interface of the at least one patient computing device 104e-h. The patient data receiving module 106 receives the entered additional medical patient data and adds it to the already present medical patient data.

If, based on the received medical patient data (including the provided additional medical patient data) and the received clinical trial data, the patient fulfils the predetermined criteria of the at least one clinical trial, the guideline adapting module 109 adapts the BCDG to provide an adapted connected directed graph (ACDG) 400 by integrating at least one additional node 401e-h, 401j-k into the BCDG. After adapting the BCDG to provide the ACDG 400 the output module 110 outputs the ACDG 400 to the medical professional graphical user interface on the at least one medical professional computing device 104a-d and additionally or alternatively to the patient graphical user interface on the at least one patient computing device 104e-h. The ACDG 400 may also be continuously adapted by adding or replacing nodes or by adding or replacing medical actions to nodes if new options for further treatment of the patient, for example new clinical trials, are available.

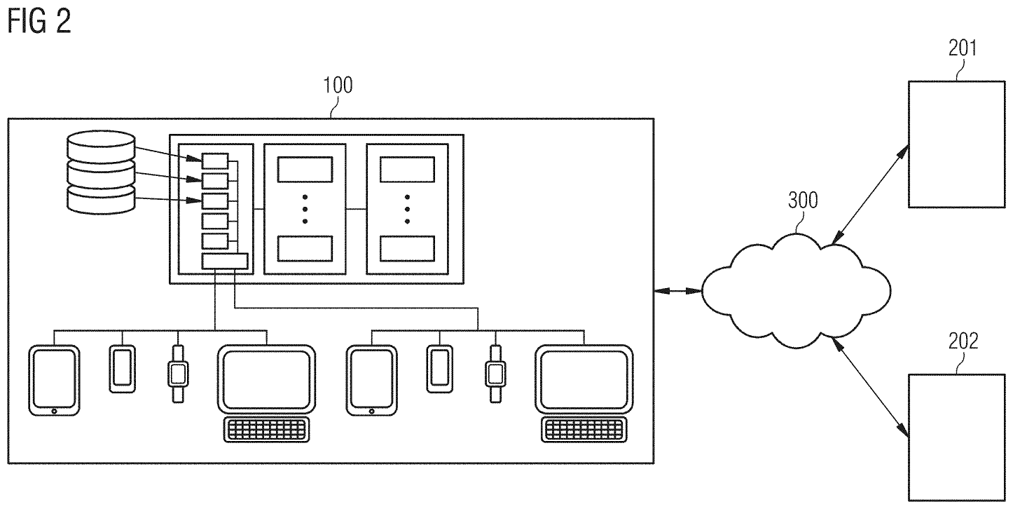

FIG. 2 shows a schematic diagram illustrating a medical recommendation system 100 according to another example.

The medical recommendation system 100 in FIG. 2 comprises the features described in the description of FIG. 1 and is connected over a network 300 to a first remote computing device 201 and a second remote computing device 202. The medical recommendation system 100 may for example be located at a first hospital. The first remote computing device 201 may for example be located a second hospital and the second remote computing device 202 may for example be located at a third hospital. The medical recommendation system 100 may for example communicate over the network 300 with the first remote computing device 201 and the second remote computing device 202 to send and additionally or alternatively receive external data. The external data may for example be (additional) medical patient data, clinical trial data and additionally or alternatively a medical guideline in a basic version or in an adapted version. Based on the external data, the received medical patient data and the received clinical trial data the medical recommendation system 100 may be able to adapt the BCDG or the ACDG 400 and may therefore be able to present (new) eligible clinical trials or potentially eligible clinical trials to the medical professional via the medical professional graphical user interface on the at least one medical professional computing device 104a-d and additionally or alternatively to the patient via the patient graphical user interface on the at least one patient computing device 104e-h. In this way the ACDG 400 may continuously be updated if other hospitals have access to other/new clinical trials.

FIG. 3 shows an adapted connected directed graph (ACDG) 400. The ACDG 400 comprises a plurality of nodes 401a-e and a plurality of directed edges 402a-d connecting the plurality of nodes 401a-e. The plurality of nodes 401a-e represent medical actions and the plurality of edges 402a-d represent options, and additionally or alternatively possibilities.

In the example illustrated in FIG. 3 the plurality of nodes 401a-d represent a basic connected directed graph, BCDG, wherein the BCDG represents a medical guideline in a basic version. In this example the patient fulfilled predetermined criteria of at least one clinical trial. The at least one clinical trial is represented by the node 401e. The BCDG is connected to the node 401e via a directed edge 402b. A combination of the BCDG with the node 401e that represents the at least one clinical trial and the directed edge 402b is the ACDG 400 representing the adapted medical guideline.

In FIG. 3 a current position (or: state, or: status) of the patient in the ACDG 400 is indicated by an icon showing a stylized human figure at node 401a. At the current position at node 401a a medical professional and additionally or alternatively the patient only has one single option represented by edge 402a that is a medical action represented by node 401b.

If the patient in FIG. 3 changes the current position (or state, or status) from the node 401a to the node 401b the medical professional and additionally or alternatively the patient are able to choose between a first medical action represented by node 401c, a second medical action represented by node 401d and the clinical trial represented by node 401e.

FIG. 4 shows another ACDG 400 of a patient. The ACDG 400 in FIG. 4 comprises the plurality of nodes 401a-e and the plurality of directed edges 402a-d of FIG. 3 and additionally the node 401f, directed edge 402e and directed edge 402f. The node 401f represents an additional medical action to test an eligibility of the patient for the at least one clinical trial represented by node 401e.

In FIG. 4 the current position (or state, or status) of the patient in the ACDG 400 is indicated by the icon showing the human figure at the node 401a. At the current position at node 401a the medical professional and additionally or alternatively the patient has two options to choose. A first option at the node 401a is the medical action represented by node 401b. A second option at the node 401a is the additional medical action represented by the node 401f.

If in FIG. 4 the medical professional and additionally or alternatively the patient decide to choose the additional medical action represented by the node 401f, the current position (or state, or status) of the patient changes to the node 401f. With the current position at the node 401f the medical professional and additionally or alternatively the patient can only choose the medical action represented by the node 401b. After completing the additional medical action represented by the node 401f the current position of the patient changes from the node 401f to the node 401b. If the result of the additional medical action represented by node 401f, for example a blood test, indicates that the patient is eligible for the at least one clinical trial represented by the node 401e the medical professional and additionally or alternatively the patient have the option to choose between the first medical action represented by the node 401c, the second medical action represented by the node 401d and, due to the result of the additional medical action represented by node 401f, the clinical trial represented by the node 401e.

FIG. 5 shows yet another ACDG 400. The ACDG in FIG. 5 comprises the plurality of nodes 401a-d and the plurality of directed edges 402a, 402c and 402d of FIG. 3 and additionally the node 401g connected to node 401b via directed edge 402g and directed edge 402h as well as node 401h connected to node 401b via directed edge 402i. The node 401g represents an additional medical action to test an eligibility of the patient for at least one clinical trial represented by the node 401h.

In FIG. 5 the current position of the patient in the ACDG 400 is indicated by the icon showing the human figure at the node 401b. With the current position at the node 401b the medical professional and additionally or alternatively the patient are able to choose between the first medical action represented by node 401c, the second medical action represented by the node 401d and the additional medical action represented by the node 401g. If the medical professional and additionally or alternatively the patient decide to choose the additional medical action represented by the node 401g the current position of the patient changes from the node 401b to the node 401g. If the additional medical action represented by the node 401g, for example an MRI examination, indicates that the patient is eligible for the at least one clinical trial represented by the node 401h then the patient is able to choose the at least one clinical trial. If the additional medical action represented by the node 401g indicates that the patient is not eligible for the at least one clinical trial represented by the node 401h then the current position of the patient changes from node 401g back to 401b along edge 402g. Back at position 401b the medical professional and additionally or alternatively the patient are able to choose between the first medical action represented by node 401c and the second medical action represented by the node 401d. In this situation, the edges 402g, 402h, 402i and the nodes 401g, 401h may be automatically removed from the ACDG 400 as they no longer represent valid options. Alternatively, they may be rendered graphically distinct from available (or: valid) option such as the ones represented by nodes 401c, 401d; for example, edges 402g, 402h, 402i and the nodes 401g, 401h may be displayed in a different tone or color (e.g. in gray as compared to blue of the other edges and nodes, or in red as compared to green of the other edges and nodes and/or the like).

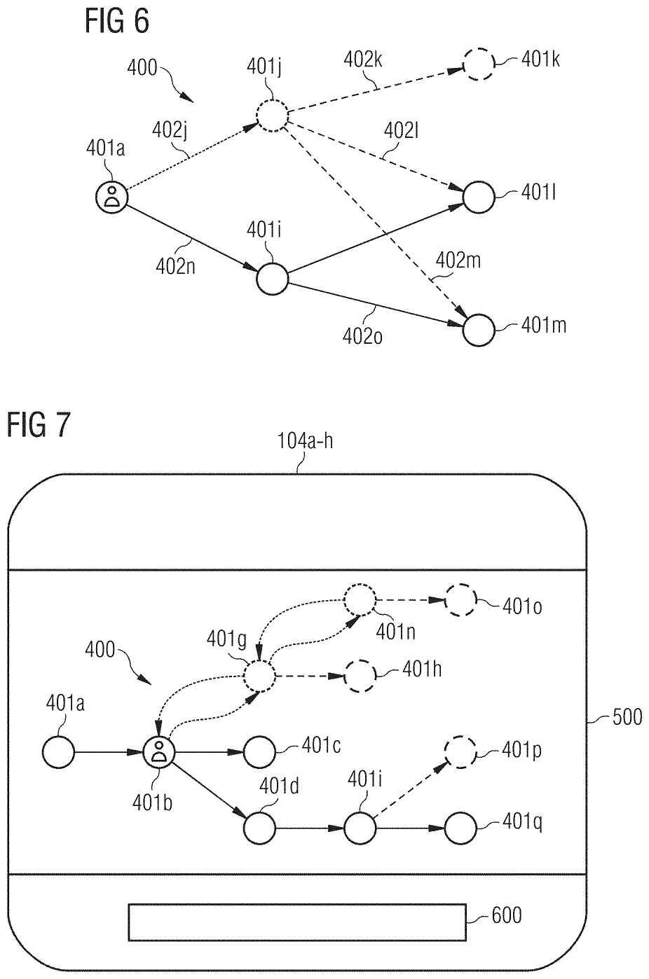

FIG. 6 shows yet another ACDG 400. The ACDG 400 of FIG. 6 comprises node 401a, node 401j, node 401k, node 401i, node 401l and node 401m connected by directed edge 402j, directed edge 402k, directed edge 402l, directed edge 402m, directed edge 402n and directed edge 402o. In FIG. 6 the node 401j is a modified node. The modified node represents a medical action, for example a blood test. The blood test may test example blood values a, b and c. In order to decide whether the patient is eligible for the clinical trial represented by the node 401k an additional blood test result of blood test d is necessary. Thus, the node 401j has been modified by adding the medical step blood test d. In case the result of the blood test d is such that the patient may enter the clinical trial, the medical professional and/or patient can choose to proceed to the node 401k representing the clinical trial or to proceed to one of the nodes 401l, 401m that would have been accessible without performing the blood tests according to the modified node 401j (via the node 401i which could represent the original set of blood tests a, b and c).

FIG. 7 shows an embodiment of a medical professional or patient computing device 104a-h as described above displaying the medical professional or patient graphical user interface. The medical professional/patient computing device 104a-h comprises, for example, a monitor 500 and a keyboard 600.

The graphical user interface is displayed on the monitor 500. The graphical user interface here exemplarily displays an ACDG 400 comprising ten nodes 401a-d, 401g-i, 401n-q and respective directed edges. The graphical user interface may further display a description of a clinical trial A represented by node 401o, a description of a clinical trial B represented by node 401h and a description of a clinical trial C represented by node 401p, necessary additional diagnostic steps or needed additional medical patient data for determining eligibility of the patient for clinical trial A or B represented by node 401n, and consequences for the patient of each clinical trial (nodes 401o, 401h and 401p) and further medical action (nodes 401c and 401q).

The further needed additional medical patient data for determining eligibility of the patient for clinical trial A or B represented by node 401n can be entered via the keyboard 600 or alternatively be provided via an interface (e.g. USB-interface) from a storage device (e.g. USB-stick).

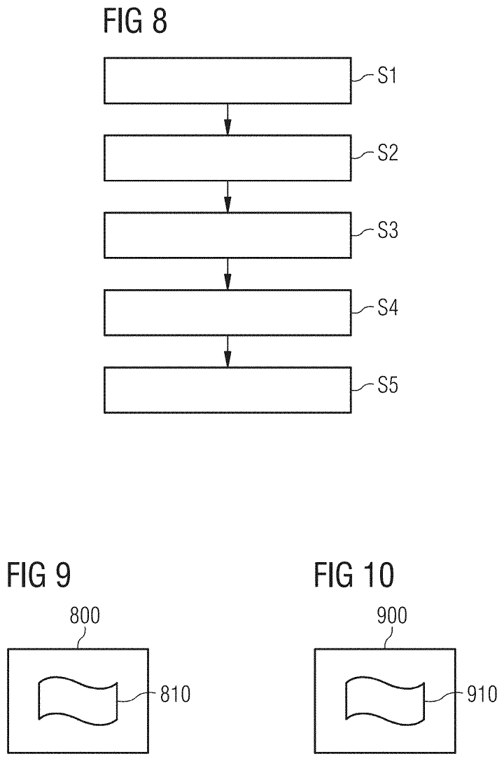

FIG. 8 shows a schematic flow diagram illustrating a method according to an embodiment of the second embodiment of the present invention. The method may be implemented by the medical recommendation system 100 of FIG. 1 and comprises the steps of receiving S1 medical patient data, receiving S2 clinical trial data, receiving S3 a medical guideline, determining S4 whether the patient fulfils predetermined criteria of at least one clinical trial and outputting S5 the ACDG 400.

In the step S1 medical patient data pertaining to a patient is received, for example from an external data storage like a hospital management system.

In the step S2 clinical trial data is received, for example from an external data storage of a site conducting the clinical trial.

In the step S3 the medical guideline in a basic version is received. The medical guideline is represented by a basic connected directed graph (BCDG). The BCDG comprises a plurality of nodes representing medical actions and a plurality of directed edges representing options/possibilities. The medical guideline in a basic version may for example be received from the hospital management system.

In the step S4 it is determined whether the patient fulfils predetermined criteria of at least one clinical trial. The determination is based on the received medical patient data and the received clinical trial data. If the patient fulfils the predetermined criteria of the at least one clinical trial, the BCDG is adapted to provide an adapted connected directed graph (ACDG) 400. Thereto the BCDG is adapted into the ACDG 400 by integrating at least one additional node into the BCDG.

In the step S5 the ACDG 400 is output, for example to a medical professional or patient computing device where it is displayed on a medical professional/patient graphical user interface.

FIG. 9 schematically illustrates a computer program product 800 comprising executable program code 810 configured to, when executed, perform the method according to the second embodiment of the present invention, in particular as has been described with respect to the previous figures.

FIG. 10 schematically illustrates a computer-readable data carrier 900 comprising executable program code 910 configured to, when executed, perform the method according to the second embodiment of the present invention, in particular as has been described with respect to the previous figures.

Although specific embodiments have been illustrated and described herein, it will be appreciated by those of ordinary skill in the art that a variety of alternate and/or equivalent implementations exist. It should be appreciated that the example embodiment or example embodiments are only examples, and are not intended to limit the scope, applicability, or configuration in any way. Rather, the foregoing summary and detailed description will provide those skilled in the art with a convenient road map for implementing at least one example embodiment, it being understood that various changes may be made in the function and arrangement of elements described in an example embodiment without departing from the scope as set forth in the appended claims and their legal equivalents. Generally, this application is intended to cover any adaptations or variations of the specific embodiments discussed herein.

In the foregoing detailed description, various features are grouped together in one or more examples for the purpose of streamlining the disclosure. It is understood that the above description is intended to be illustrative, and not restrictive. It is intended to cover all alternatives, modifications and equivalents as may be included within the scope of the invention. Many other examples will be apparent to one skilled in the art upon reviewing the above specification.

Of course, the embodiments of the method according to the invention and the imaging apparatus according to the invention described here should be understood as being example. Therefore, individual embodiments may be expanded by features of other embodiments. In particular, the sequence of the method steps of the method according to the invention should be understood as being example. The individual steps can also be performed in a different order or overlap partially or completely in terms of time.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

Specific nomenclature used in the foregoing specification is used to provide a thorough understanding of the invention. However, it will be apparent to one skilled in the art in light of the specification provided herein that the specific details are not required in order to practice the invention. Thus, the foregoing descriptions of specific embodiments of the present invention are presented for purposes of illustration and description. They are not intended to be exhaustive or to limit the invention to the precise forms disclosed; obviously many modifications and variations are possible in view of the above teachings. The embodiments were chosen and described in order to best explain the principles of the invention and its practical applications, to thereby enable others skilled in the art to best utilize the invention and various embodiments with various modifications as are suited to the particular use contemplated. Throughout the specification, the terms “including” and “in which” are used as the plain-English equivalents of the respective terms “comprising” and “wherein,” respectively. Moreover, the terms “first,” “second,” and “third,” etc., are used merely as labels, and are not intended to impose numerical requirements on or to establish a certain ranking of importance of their objects. In the context of the present description and claims the conjunction “or” is to be understood as including (“and/or”) and not exclusive (“either . . . or”).

What is claimed is:

1. A medical recommendation system for a user, comprising:
a computing device, configured to implement at least:
a patient data receiving module configured to receive medical patient data pertaining to a patient;
a clinical trial data receiving module configured to receive clinical trial data;
a medical guideline receiving module, configured to receive a medical guideline in a basic version, the medical guideline being represented by a basic connected directed graph (BCDG) including a plurality of nodes representing medical actions and a plurality of directed edges representing at least one of options and possibilities;
a guideline adapting module configured to determine whether the patient fulfils criteria of at least one clinical trial based on the medical patient data received and the clinical trial data received, and configured to adapt the BCDG, upon the patient being determined to have fulfilled the criteria of the at least one clinical trial, to provide an adapted connected directed graph (ACDG) by integrating at least one additional node into the BCDG; and
an output module configured to output the ACDG.

2. The medical recommendation system of claim 1, further comprising:
at least one medical professional graphical user interface configured to display at least a part of the ACDG to a medical professional.

3. The medical recommendation system of claim 1, further comprising:
at least one patient graphical user interface configured to display at least a part of the ACDG to the patient.

4. The medical recommendation system of claim 1, wherein at least one node of the at least one additional node represents an additional medical action to test an eligibility of the patient for the at least one clinical trial.

5. The medical recommendation system of claim 1, wherein at least one node of the at least one additional node represents the at least one clinical trial.

6. The medical recommendation system of claim 1, wherein at least one node of the plurality of nodes representing the medical actions includes a sequence of medical steps, each of the medical steps representing an interaction of the medical professional with at least one of a medical device and a patient, and wherein the guideline adapting module is configured to modify at least one node of the plurality of nodes by at least one of adding at least one further medical step to and replacing at least one medical step in the sequence of medical steps.

7. The medical recommendation system of claim 2, wherein the guideline adapting module is configured to generate an examination request signal indicating that at least one additional medical examination of the patient is required to identify an eligible clinical trial and wherein the output module is configured to output the examination request signal to the at least one medical professional graphical user interface.

8. The medical recommendation system of claim 2, wherein the computing device further implements an information request module, configured to generate an information request signal indicating that additional medical patient data is required and wherein the output module is configured to output the information request signal to the at least one medical professional graphical user interface.

9. The medical recommendation system of claim 8, wherein the additional medical patient data comprises at least one of:
information about a medical history of the patient;
information about pre-existing medical conditions of the patient,
information indicating a distance the patient is willing to travel to participate in a clinical trial, and
information indicating a preferred type of therapy.

10. The medical recommendation system of claim 8, wherein the at least one patient graphical user interface is configured to enable the patient to enter the additional medical patient data, wherein the patient data receiving module is configured to receive the additional medical patient data and to add the entered additional medical patient data to the medical patient data.

11. A method for providing a medical recommendation to a user, comprising:
receiving medical patient data pertaining to a patient;
receiving clinical trial data;
receiving a medical guideline in a basic version, the medical guideline being represented by a basic connected directed graph (BCDG) including a plurality of nodes representing medical actions and a plurality of directed edges representing at least one of options and possibilities;
determining whether the patient fulfils criteria of at least one clinical trial based on the medical patient data received and the clinical trial data received and adapting the BCDG, upon the determining indicating that the patient fulfils the criteria of the at least one clinical trial, to provide an adapted connected directed graph (ACDG) by integrating at least one additional node into the BCDG; and
outputting the ACDG.

12. A non-transitory computer program product storing executable program code which, when executed by a computer, cause the computer to carry out the method of claim 11.

13. A non-transitory computer-readable data carrier storing executable program code which, when executed by a computer, cause the computer to carry out the method of claim 11.

14. The medical recommendation system of claim 2, further comprising:
at least one patient graphical user interface configured to display at least a part of the ACDG to the patient.

15. The medical recommendation system of claim 2, wherein at least one node of the at least one additional node represents an additional medical action to test an eligibility of the patient for the at least one clinical trial.

16. The medical recommendation system of claim 2, wherein at least one node of the at least one additional node represents the at least one clinical trial.

17. The medical recommendation system of claim 2, wherein at least one node of the plurality of nodes representing the medical actions includes a sequence of medical steps, each of the medical steps representing an interaction of the medical professional with at least one of a medical device and a patient, and wherein the guideline adapting module is configured to modify at least one node of the plurality of nodes by at least one of adding at least one further medical step to and replacing at least one medical step in the sequence of medical steps.

18. The medical recommendation system of claim 3, wherein the guideline adapting module is configured to generate an examination request signal indicating that at least one additional medical examination of the patient is required to identify an eligible clinical trial and wherein the output module is configured to output the examination request signal to at least one of the at least one medical professional graphical user interface and the at least one patient graphical user interface.

19. The medical recommendation system of claim 3, wherein the computing device further implements an information request module, configured to generate an information request signal indicating that additional medical patient data is required and wherein the output module is configured to output the information request signal to at least one of the at least one medical professional graphical user interface and the at least one patient graphical user interface.
